(19) United States
(12) Patent Application Publication
(10) Pub. No.: US 2025/0189427 A1
(43) Pub. Date: Jun. 12, 2025

(54) EXPERIMENTAL APPARATUS AND METHOD FOR SIMULATING FRACTURING OF FAN-SHAPED WELL PATTERN

(71) Applicant: China University of Petroleum-Beijing, Beijing City (CN)

(72) Inventors: Guangqing ZHANG, Beijing City (CN); Shuai LI, Beijing City (CN); Haode CHENG, Beijing City (CN)

(73) Assignee: China University of Petroleum-Beijing, Beijing City (CN)

(21) Appl. No.: 18/960,463

(22) Filed: Nov. 26, 2024

(30) Foreign Application Priority Data
Dec. 11, 2023 (CN) 202311687852.8

(51) Int. Cl. G01N 15/08; G01N 33/24

(52) U.S. Cl. CPC G01N 15/082; G01N 33/24

(57) ABSTRACT

The present disclosure provides an experimental apparatus and method for simulating fracturing of a fan-shaped well pattern. The apparatus includes a stratum simulator and a wellbore simulator group. The stratum simulator includes a chamber into which materials are poured to simulate a stratum environment. The wellbore simulator group at least includes a first wellbore simulator and a second wellbore simulator. A first end of the first wellbore simulator and a first end of the second wellbore simulator are respectively located outside the chamber and provided with a control valve. A second end of the first wellbore simulator is extended into the chamber and provided with a first perforation simulator, a second end of the second wellbore simulator is extended into the chamber and passes through the corresponding first perforation simulator, and the second end of the second wellbore simulator is provided with a second perforation simulator.

### CROSS-REFERENCE TO RELATED APPLICATION

This application claims priority to Chinese Patent Application No. 202311687852.8, filed on Dec. 11, 2023, which is hereby incorporated by reference in its entirety.

### TECHNICAL FIELD

The present disclosure relates to the field of petroleum engineering rock mechanics, and particularly to an experimental apparatus and method for simulating fracturing of a fan-shaped well pattern.

### BACKGROUND

Production enhancement techniques such as multi-stage fracturing are often used in hydraulic fracturing of tight and low permeability reservoir rocks such as shale. At present, the main understanding of fracture initiation and propagation is that the principal stress direction controls the orientation of the fracture. Therefore, in the practical engineering, horizontal well patterns are designed and constructed based on such understanding, and horizontal wells are parallel to each other. Then, each horizontal well is fractured in stages to form a complex fracture network, so as to reduce the construction cost and maximize the use of reservoir reserves.

Multi-stage fracturing of the horizontal wells is one of the key techniques for the efficient development of unconventional oil and gas reservoirs, but some well sites are limited by the factors such as reservoir distribution area and environmental protection, and it is impossible to carry out a conventional well arrangement. Therefore, in order to maximize the use of such kind of reservoir reserves, a well arrangement mode such as the fan-shaped well pattern may be employed to form a fracture network. However, no appropriate experimental device is available for the well layout of the fan-shaped well pattern, making it impossible to conduct fracturing experiments for the fan-shaped well pattern, and there is no theoretical guidance for the actual fracturing operation.

Therefore, based on years of experience and practice in related industries, the inventor proposes an experimental apparatus and method for simulating fracturing of a fan-shaped well pattern to overcome the defects of the prior art.

### SUMMARY

The present disclosure aims to provide an experimental apparatus and method for simulating fracturing of a fan-shaped well pattern, which can improve and refine the existing fracturing and layout of the fan-shaped well pattern, and provide more experimental basis and theoretical guidance for actual oilfield fracturing and researches on the fracturing design of the fan-shaped well pattern.

The objective of the present disclosure can be achieved by the following solutions.

The present disclosure provides an experimental apparatus for simulating fracturing of a fan-shaped well pattern, including a stratum simulator and a wellbore simulator group. The stratum simulator includes a chamber into which materials are poured to simulate a stratum environment. The wellbore simulator group at least includes a first wellbore simulator and a second wellbore simulator. A first end of the first wellbore simulator and a first end of the second wellbore simulator are respectively located outside the chamber and respectively provided with a control valve. A second end of the first wellbore simulator is extended into the chamber and provided with a first perforation simulator, a second end of the second wellbore simulator is extended into the chamber and passes through the corresponding first perforation simulator, and the second end of the second wellbore simulator is provided with a second perforation simulator, so as to simulate a multi-stage fracturing scenario of the fan-shaped well pattern.

In an exemplarily embodiment of the present disclosure, there are a plurality of the wellbore simulator groups which are independent from each other.

In an exemplarily embodiment of the present disclosure, an included angle between the first wellbore simulator and a maximum horizontal principal stress direction and an included angle between the second wellbore simulator and the maximum horizontal principal stress direction are preset to simulate different multi-stage fracturing scenarios of the fan-shaped well pattern.

In an exemplarily embodiment of the present disclosure, the control valve is a three-way valve, which comprises a first port, a second port and a third port, which are respectively connected to a booster pump, the wellbore simulator group and a pressure gauge.

In an exemplarily embodiment of the present disclosure, the first perforation simulator and the second perforation simulator each is formed by two polytetrafluoroethylene sheets that are oppositely attached to each other, the first perforation simulator is nested at the second end of the first wellbore simulator, and the second perforation simulator is nested at the second end of the second wellbore simulator.

In an exemplarily embodiment of the present disclosure, a direction of the first perforation simulator relative to the first wellbore simulator is adjusted, and/or a direction of the second perforation simulator relative to the second wellbore simulator is adjusted, to change an angle of a perforation formed by two polytetrafluoroethylene sheets.

The present disclosure provides an experimental method for simulating fracturing of a fan-shaped well pattern, which adopts the experimental apparatus for simulating fracturing of a fan-shaped well pattern described above, the method including:
step S1: applying a three-dimensional stress confining pressure to the materials poured into the stratum simulator to simulate a stratum pressure; and
step S2: sequentially injecting high-pressure fluid into the first wellbore simulator and the second wellbore simulator in the wellbore simulator group for fracturing, respectively, to simulate a multi-stage fracturing scenario of the fan-shaped well pattern in a stratum.

In an exemplarily embodiment of the present disclosure, in the step S1, the wellbore simulator group is connected to a booster pump and a pressure gauge through a control valve, and the simulated stratum pressure is adjusted through a cooperation of the booster pump and the wellbore simulator group, to make the simulated stratum pressure the same as an actual stratum pressure.

In an exemplarily embodiment of the present disclosure, in the step S2, the multi-stage fracturing scenario of the fan-shaped well pattern in the stratum is simulated using a synchronous fracturing mode, the number of the wellbore simulator groups is at least two, and the synchronous fracturing mode includes:
step S201: injecting high-pressure fluid into the first wellbore simulators in the two wellbore simulator groups for fracturing, respectively;
step S202: collecting fracturing data of the two first wellbore simulators respectively, and stopping injecting the high-pressure fluid into the two first wellbore simulators;
step S203: injecting high-pressure fluid into the second wellbore simulators of the two wellbore simulator groups for fracturing, respectively;
step S204: collecting fracturing data of the two second wellbore simulators, respectively, and stopping injecting the high-pressure fluid into the two second wellbore simulators; and
step S205: completing the multi-stage fracturing simulation experiment on the fan-shaped well pattern in the stratum using the synchronous fracturing mode.

In an exemplarily embodiment of the present disclosure, in the step S2, the multi-stage fracturing scenario of the fan-shaped well pattern in the stratum is simulated using a zipper fracturing mode, the number of the wellbore simulator groups is at least two, and the zipper fracturing mode includes:
step S201′: injecting high-pressure fluid into the first wellbore simulator in the first wellbore simulator group for fracturing, collecting fracturing data of the first wellbore simulator, and then stopping injecting the high-pressure fluid;
step S202′: injecting high-pressure fluid into the first wellbore simulator in the second wellbore simulator group for fracturing, collecting fracturing data of the first wellbore simulator, and then stopping injecting the high-pressure fluid;
step S203′: injecting high-pressure fluid into the second wellbore simulator in the first wellbore simulator group for fracturing, collecting fracturing data of the second wellbore simulator, and then stopping injecting the high-pressure fluid;
step S204′: injecting high-pressure fluid into the second wellbore simulator in the second wellbore simulator group for fracturing, and then stopping injecting the high-pressure fluid; and
Step S205′: completing the multi-stage fracturing simulation experiment on the fan-shaped well pattern in the stratum using the zipper fracturing mode.

As can be seen from the above description, the experimental apparatus and method for simulating fracturing of a fan-shaped well pattern according to the present disclosure have the following characteristics and advantages: materials are poured into the chamber of the stratum simulator to simulate the stratum environment; the wellbore simulator group at least includes a first wellbore simulator and a second wellbore simulator, a first end of the first wellbore simulator and a first end of the second wellbore simulator are respectively located outside the chamber and respectively provided with a control valve to control the injection of high-pressure fluid, a second end of the first wellbore simulator is extended into the chamber and provided with a first perforation simulator, a second end of the second wellbore simulator is extended into the chamber and passes through the corresponding first perforation simulator, and the second end of the second wellbore simulator is provided with a second perforation simulator, thereby forming the fan-shaped well pattern layout, and realizing realistic simulation of the multi-stage fracturing scenario of the fan-shaped well pattern. By means of the experimental apparatus, it is possible to improve and refine the existing fracturing and layout of the fan-shaped well pattern, and provide more experimental basis and theoretical guidance for actual oilfield fracturing and researches on the fracturing design of the fan-shaped well pattern.

### BRIEF DESCRIPTION OF DRAWINGS

The following drawings are intended only to schematically illustrate and explain the present disclosure and do not limit the scope of the present disclosure. In the drawings,

FIG. 1A illustrates a structural diagram of an experimental apparatus for simulating fracturing of a fan-shaped well pattern according to the present disclosure.

FIG. 1B illustrates a structural diagram of the first perforation simulator and the second perforation simulator according to an embodiment of the present disclosure;

FIG. 1C illustrates a schematic exploded view of the first perforation simulator or the second perforation simulator according to an embodiment of the present disclosure;

FIG. 2 illustrates a structural diagram of an experimental apparatus for simulating fracturing of a fan-shaped well pattern according to a specific embodiment of the present disclosure.

FIG. 3 illustrates a process flowchart of an experiment method for simulating fracturing of a fan-shaped well pattern according to an embodiment of the present disclosure.

FIG. 4 illustrates a process flowchart of an experiment method for simulating fracturing of a fan-shaped well pattern according to another embodiment of the present disclosure.

FIG. 5 illustrates a process flowchart of an experimental method for simulating fracturing of a fan-shaped well pattern according to yet another embodiment of the present disclosure.

### REFERENCE NUMERALS IN THE DRAWINGS

1: stratum simulator; 101: chamber;
2: wellbore simulator group; 201: first wellbore simulator;
202: second wellbore simulator; 3: control valve;
300: capillary tube; 301: bend section;
401: first perforation simulator; 402: second perforation simulator;
400: sheet; 500: perforation.

### DETAILED DESCRIPTION OF EMBODIMENTS

For a clearer understanding of the technical features, objectives and effects of the present disclosure, specific embodiments of the present disclosure will now be described with reference to the drawings.

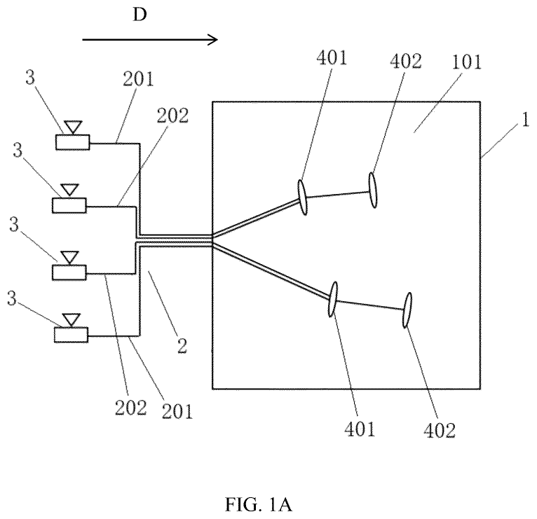

As illustrated in FIG. 1A, It is one aspect of the present disclosure to provide an experimental apparatus for simulating fracturing of a fan-shaped well pattern, which includes a stratum simulator 1 and a wellbore simulator group 2. The stratum simulator 1 includes a chamber 101 into which materials are poured to simulate a stratum environment. The wellbore simulator group at least includes a first wellbore simulator 201 and a second wellbore simulator 202. Two ends of the first wellbore simulator 201 are respectively a first end and a second end. Two ends of the second wellbore simulator 202 are respectively a first end and a second end. The first end of the first wellbore simulator 201 and the first end of the second wellbore simulator 202 are respectively located outside the chamber 101 and respectively provided with a control valve 3. The second end of the first wellbore simulator 201 is extended into the chamber 101 and provided with a first perforation simulator 401, the second end of the second wellbore simulator 202 is extended into the chamber 101 and passes through the corresponding first perforation simulator 401, and the second end of the second wellbore simulator 202 is provided with a second perforation simulator 402, so as to simulate a multi-stage fracturing scenario of a fan-shaped well pattern.

According to the present disclosure, materials are poured into the chamber 101 of the stratum simulator 1 to simulate the stratum environment, the wellbore simulator group 2 at least includes the first wellbore simulator 201 and the second wellbore simulator 202, the first ends of the first wellbore simulator 201 and the second wellbore simulator 202 are respectively located outside the chamber 101 and provided with the control valve 3 to control the injection of high-pressure fluid, the second end of the first wellbore simulator 201 is extended into the chamber 101 and provided with the first perforation simulator 401, the second end of the second wellbore simulator 202 is extended into the chamber 101 and passes through the corresponding first perforation simulator 401, and the second end of the second wellbore simulator 202 is provided with the second perforation simulator 402, thereby forming a fan-shaped well pattern and realizing a real simulation of the multi-segment fracturing scenario for the fan-shaped well pattern. The experimental apparatus of the present disclosure can improve and refine the existing fracturing and layout of the fan-shaped well pattern, and provide more experimental basis and theoretical guidance for actual oilfield fracturing and researches on the fracturing design of the fan-shaped well pattern.

In an alternative embodiment of the present disclosure, there may be a plurality of wellbore simulator groups 2 which are independent from each other. As illustrated in FIG. 1A, there may be two wellbore simulator groups 2, each of which is used to simulate one horizontal well. Each wellbore simulator group 2 includes one first wellbore simulator 201 and one second wellbore simulator 202 to simulate one horizontal well. Therefore, the first wellbore simulators 201 and the second wellbore simulators 202 in the two wellbore simulator groups 2 can simulate two horizontal wells, without any interference between the two wellbore simulator groups 2, so that multi-stage fracturing can be realized according to preset experimental conditions. The number of the wellbore simulator groups 2 may be flexibly set according to needs, and the specific number is not limited in the present disclosure.

Further, the first wellbore simulator 201 and the second wellbore simulator 202 each may be, but not limited to, a capillary tube 300 made of high-pressure resistant metal.

In an alternative embodiment of the present disclosure, the stratum simulator 1 is a prefabricated mold having the chamber 101, and the materials poured into the chamber 101 may be, but not limited to, a mixture of cement, gypsum, quartz sand and water, which is poured into the chamber 101 after being stirred. The size of the stratum simulator 1 may be flexibly set according to the experimental requirements of the simulated stratum. For example, the stratum simulator 1 may be a square container with a length, a width and a height that are all 300 mm. The portion of the wellbore simulator group 2 that needs to be disposed in the materials may be placed in the chamber 101 before the materials are poured into the chamber 101, thus eliminating the operations such as drilling, cutting, and well sticking that are performed for disposing the wellbore simulator group 2 after pouring, making the arrangement convenient and fast.

In an alternative embodiment of the present disclosure, as illustrated in FIG. 1A, an included angle between the first wellbore simulator 201 and a maximum horizontal principal stress direction and an included angle between the second wellbore simulator 202 and the maximum horizontal principal stress direction are preset to simulate different multi-stage fracturing scenarios of the fan-shaped well pattern. It should be understood that the maximum horizontal principal stress referred to in the present disclosure is a maximum horizontal stress in the stratum, being the maximum stable stress in the stratum. When laying out the first wellbore simulator 201 and the second wellbore simulator 202, it is necessary to ensure that the first wellbore simulator 201 and the second wellbore simulator 202 are not laid out in a direction perpendicular to the maximum horizontal principal stress direction D, so that the included angle between the first wellbore simulator 201 and the maximum horizontal principal stress direction D and the included angle between the second wellbore simulator 202 and the maximum horizontal principal stress direction D are not 90°. During the experiment, the included angle between the first wellbore simulator 201 and/or the second wellbore simulator 202 and the maximum horizontal principal stress direction D may be changed according to preset simulation conditions, so as to realize the simulation of the fan-shaped well pattern.

In an alternative embodiment of the present disclosure, the control valve 3 may be a three-way valve, which includes a first port, a second port and a third port, which are respectively connected to a booster pump, the wellbore simulator group 2 and a pressure gauge, so that the injection of high-pressure fluid can be controlled in real time by the booster pump and the three-way valve, and a pumping pressure can be monitored in real time by the pressure gauge. In the present disclosure, the control valve 3 may be independently connected to each of the first wellbore simulator 201 and the second wellbore simulator 202, so that each of the first wellbore simulator 201 and the second wellbore simulator 202 can be independently controlled for pumping. The number of the three-way valves may be flexibly set as needed, for example, there may be four three-way valves as shown in FIG. 1. In addition, by disposing the control valve 3, it can be independently shut off after a single stage of fracturing in the fan-shaped well pattern, so as to control the first wellbore simulator 201 or the second wellbore simulator 202 corresponding to the control valve 3 to stop injecting high-pressure fluid, thereby keeping a stable pressure in the fracture.

In an alternative embodiment of the present disclosure, the first perforation simulator 401 and the second perforation simulator 402 each may be formed by two polytetrafluoroethylene sheets that are oppositely attached to each other. The first perforation simulator 401 is nested at the second end of the first wellbore simulator 201, and the second perforation simulator 402 is nested at the second end of the second wellbore simulator 202. The perforation simulators formed in this structure can prevent the materials in the stratum simulator 1 from blocking the first wellbore simulator 201 and the second wellbore simulator 202, thereby ensuring the smooth progress of the experiment.

Further, by adjusting a direction of the first perforation simulator 401 relative to the first wellbore simulator 201, and/or by adjusting a direction of the second perforation simulator 402 relative to the second wellbore simulator 202, an angle of the perforation 500 formed by the two polytetrafluoroethylene sheets can be changed, so that an injection angle of high-pressure fluid can be controlled according to the actual experimental conditions. The polytetrafluoroethylene sheet may be a small-diameter circular sheet, which can reduce the perforation size and improve the utilization efficiency of the stratum simulator 1. The size of the polytetrafluoroethylene sheet may be flexibly set according to the actual experimental conditions. For example, the polytetrafluoroethylene sheet may have a diameter of 30 mm and a thickness of 0.5 mm. In addition, among the first perforation simulators 401 and the second perforation simulators 402, an interval between two adjacent perforation simulators may be flexibly set according to the actual experimental conditions, so that simulation experiments with different intervals can be carried out, and for example, the interval between two adjacent perforation simulators may be 80 mm.

Exemplarily, as shown in FIG. 1B and FIG. 1C, the first perforation simulator 401 includes two sheets 400, and the first wellbore simulator 201 and the second wellbore simulator 202 are each a metal capillary tube 300 having a bend section 301 that bends with respect to a main body of the metal capillary tube 300. The main body of the metal capillary tube 300 passes through one of the two sheets 400, the bent section 301 is sandwiched between the two sheets 400, and the two sheets 400 are fixed relative to each other, so that a gap is formed between the two sheets 400 in fluid communication with the outlet of the bent section 301. The fluid from the outlet of the bent section 301 can flow through the gap between the two sheets 400 and into the materials within the chamber 101 for fracturing. In other words, the fluid can flow out of the perforation 500 formed by the two sheets 400 from an annular outlet between the outer peripheral edges of the two sheets 400, and flow into the materials within the chamber 101 for fracturing.

In the example shown in FIG. 1B and FIG. 1C, the angle of the perforation 500 formed by the two sheets 400 is 90° with respect to the main body of the metal capillary tube 300. However, the angle of the perforation 500 is adjustable. For example, the angle of the perforation 500 may be adjusted by changing the angle of bending of the bent section 301 with respect to the main body of the metal capillary tube 300, thereby realizing the adjustment of the direction of the first perforation simulator 401 or the second perforation simulator 402 relative to the first wellbore simulator 201 or the second wellbore simulator 202, so as to change the fluid injection angle.

The experimental apparatus for simulating fracturing of a fan-shaped well pattern according to the present disclosure has the following characteristics and advantages:

First, in the experimental apparatus for simulating fracturing of a fan-shaped well pattern, the stratum simulator 1 may be prepared using a pouring method, and a plurality of wellbore simulator groups 2 may be placed therein, so that the fracturing of each wellbore simulator group 2 can be controlled independently for the realistic simulation of the fracturing scenario of the fan-shaped well pattern.

Second, in the experimental apparatus for simulating fracturing of a fan-shaped well pattern, the preset angle between the wellbore simulator groups 2 and the maximum horizontal principal stress direction can be changed, and neither the first wellbore simulator 201 nor the second wellbore simulator 202 is deployed in a direction perpendicular to the maximum horizontal principal stress direction, so as to simulate the fracturing scenario of the fan-shaped well pattern.

Third, in the experimental apparatus for simulating fracturing of a fan-shaped well pattern, a plurality of wellbore simulator groups 2 may be disposed to simulate a plurality of horizontal wells to achieve the multi-well and multi-fracture simulation of the fan-shaped well pattern, and the included angles between the simulated horizontal wells may be changed to meet the simulations of different sectors of the fan-shaped well pattern.

Fourth, in the experimental apparatus for simulating fracturing of a fan-shaped well pattern, it is possible to carry out the realistic simulation of the fracturing scenario of the fan-shaped well pattern, so as to improve and refine the existing fracturing and layout of the fan-shaped well pattern, and provide more experimental basis and theoretical guidance for actual oilfield fracturing and researches on the fracturing design of the fan-shaped well pattern.

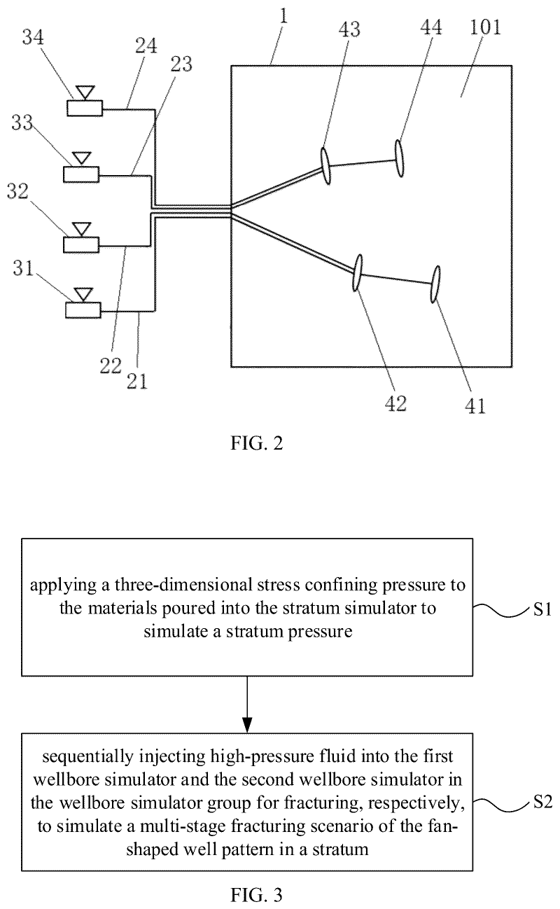

As illustrated in FIG. 3, it is another aspect of the present disclosure to provide an experimental method for simulating fracturing of a fan-shaped well pattern, which adopts the experimental apparatus for simulating fracturing of a fan-shaped well pattern described above, and the experimental method includes:
step S1: applying a three-dimensional stress confining pressure to materials poured into the stratum simulator 1 to simulate a stratum pressure; and
step S2: sequentially injecting high-pressure fluid into the first wellbore simulator 201 and the second wellbore simulator 202 in the wellbore simulator group 2, so that the high-pressure fluid fractures the materials poured into a chamber 101 through corresponding first perforation simulator 401 and second perforation simulator 402, to simulate a multi-stage fracturing scenario of the fan-shaped well pattern in a stratum.

In an alternative embodiment of the present disclosure, in the step S1, the wellbore simulator group 2 is connected to a booster pump and a pressure gauge through a control valve 3, and the simulated stratum pressure is adjusted through a cooperation of the booster pump and the wellbore simulator group 2, to make the simulated stratum pressure the same as an actual stratum pressure.

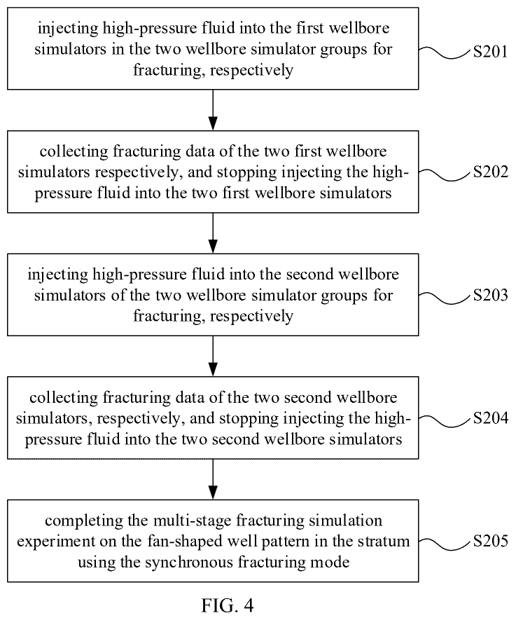

In an alternative embodiment of the present disclosure, as illustrated in FIG. 4, in the step S2, the multi-stage fracturing scenario of the fan-shaped well pattern in the stratum may be simulated using a synchronous fracturing mode, the number of the wellbore simulator groups 2 is at least two, and the synchronous fracturing mode includes:
step S201: injecting high-pressure fluid into the first wellbore simulators 201 in the two wellbore simulator groups 2, respectively, so that the high-pressure fluid fractures the materials poured into the chamber 101 via corresponding first perforation simulator 401 and second perforation simulator 402, respectively;
step S202: collecting fracturing data of the two first wellbore simulators 201 respectively, and stopping injecting the high-pressure fluid into the two first wellbore simulators 201;
step S203: injecting high-pressure fluid into the second wellbore simulators 202 of the two wellbore simulator groups 2, so that the high-pressure fluid fractures the materials poured in the chamber 101 via the corresponding second perforation simulators 402;
step S204: collecting fracturing data of the two second wellbore simulators 202, respectively, and stopping injecting the high-pressure fluid into the two second wellbore simulators 202; and
step S205: completing the multi-stage fracturing simulation experiment on the fan-shaped well pattern in the stratum using the synchronous fracturing mode.

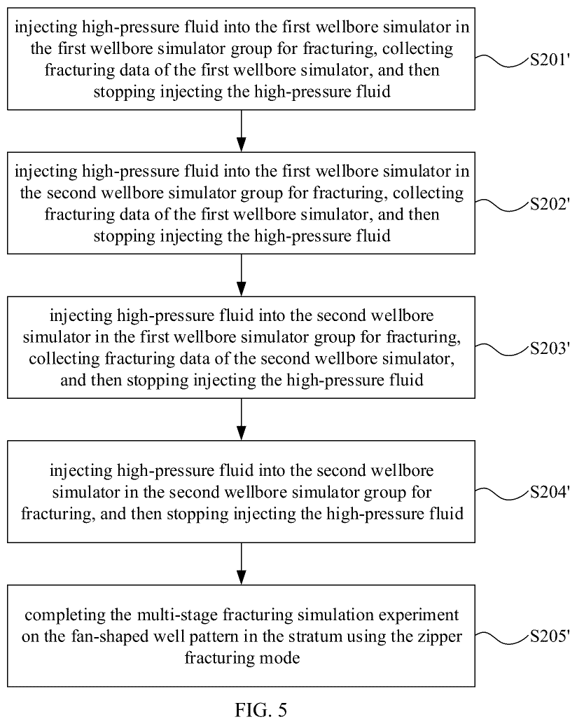

In another alternative embodiment of the present disclosure, as illustrated in FIG. 5, in the step S2, the multi-stage fracturing scenario of the fan-shaped well pattern in the stratum is simulated using a zipper fracturing mode, the number of the wellbore simulator groups 2 is at least two, and the zipper fracturing mode includes:
step S201′: injecting high-pressure fluid into the first wellbore simulator 201 in the first wellbore simulator group 2, so that the high-pressure fluid fractures materials poured into the chamber 101 via corresponding first perforation simulator 401, collecting fracturing data of the first wellbore simulator 201, and then stopping injecting the high-pressure fluid;
step S202′: injecting high-pressure fluid into the first wellbore simulator 201 in the second wellbore simulator group 2, so that the high-pressure fluid fractures the materials poured into the chamber 101 via the corresponding first perforation simulator 401, collecting fracturing data of the first wellbore simulator 201, and then stopping injecting the high-pressure fluid;
step S203′: injecting high-pressure fluid into the second wellbore simulator 202 in the first wellbore simulator group 2, so that the high-pressure fluid fractures the materials poured into the chamber 101 via the corresponding second perforation simulator 402, collecting fracturing data of the second wellbore simulator 202, and then stopping injecting the high-pressure fluid;
step S204′: injecting high-pressure fluid into the second wellbore simulator 202 in the second wellbore simulator group 2, so that the high-pressure fluid fractures the materials poured into the chamber 101 via the corresponding second perforation simulator 402, collecting fracturing data of the second wellbore simulator 202, and then stopping injecting the high-pressure fluid; and
Step S205′: completing the multi-stage fracturing simulation experiment on the fan-shaped well pattern in the stratum using the zipper fracturing mode.

In the above steps, the collected fracturing data may be, but not limited to, conventional data for carrying out a stratum fracturing experiment, such as a fracture broken pressure, a porosity, a type and rheological property of fracturing fluid, power of a fracturing device, a displacement of pumped fluid, etc. The present disclosure is intended to limit the method of simulation experiment, rather than limiting the category of the fracturing data collected.

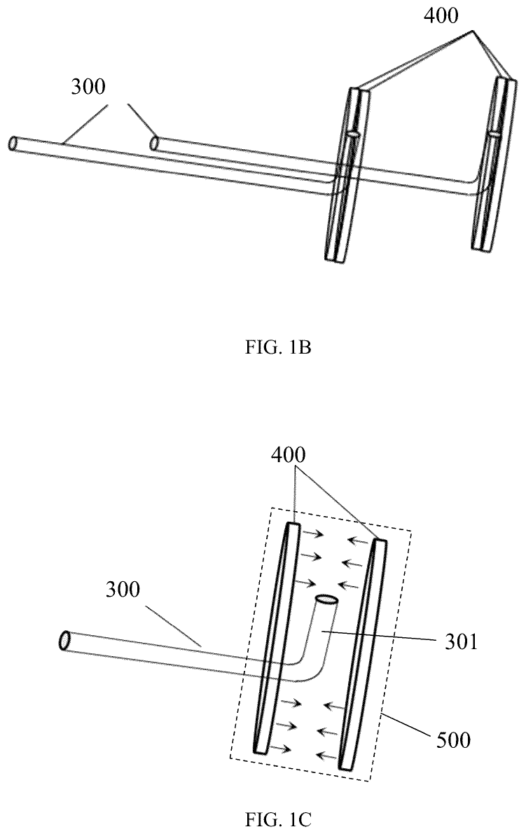

In a specific embodiment of the present disclosure, as illustrated in FIG. 2, the preparation of the experimental apparatus is as follows: two polytetrafluoroethylene sheets are oppositely attached to each other to form a perforation, and are connected to one end of a simulated wellbore by nesting, and the other end of the simulated wellbore is connected to the control valve 3.

Specifically, a second control valve 32 is connected to one end of a second wellbore 22 of a first horizontal well, and the other end of the second wellbore 22 of the first horizontal well is nested with a second perforation 42 of the first horizontal well; a first control valve 31 is connected to one end of a first wellbore 21 of the first horizontal well, the first wellbore 21 of the first horizontal well passes through the second perforation 42 of the first horizontal well, and the other end of the first wellbore 21 of the first horizontal well is nested with a first perforation 41 of the first horizontal well; a third control valve 33 is connected to one end of a second wellbore 23 of a second horizontal well, and the other end of the second wellbore 23 of the second horizontal well is nested with a second perforation 43 of the second horizontal well; a fourth control valve 34 is connected to one end of a first wellbore 24 of the second horizontal well, the first wellbore 24 of the second horizontal well passes through the second perforation 43 of the second horizontal well, and the other end of the first wellbore 24 of the second horizontal well is nested with a first perforation 44 of the second horizontal well.

The four wellbores, which have been connected respectively, form a plane (i.e., the four wellbores are in the same plane) and are placed at a central position in the stratum simulator 1, and the spacing between the adjacent perforations may be controlled to be equal during placement. Cement, gypsum, quartz sand, and water are mixed and stirred and then poured into the stratum simulator 1 to obtain the simulated stratum. The materials need to be cured in the stratum simulator 1 for a period of time, which may be, but not limited to, 14 days.

The simulation by the experimental method is as follows: after the stratum simulator 1 is prepared and cured by the above method, a three-dimensional stress confining pressure is applied to the stratum simulator 1 to simulate a stratum pressure; the other two ports of the control valve 3 are respectively connected to a booster pump and a pressure gauge, and high-pressure fluid is injected into the corresponding wellbores under the confining pressure condition while a pumping pressure is monitored.

The synchronous fracturing mode comprises:
simultaneously injecting high-pressure fluid into the first wellbore 21 of the first horizontal well and the first wellbore 24 of the second horizontal well for fracturing, and collecting fracturing data, and after the fracturing is completed, stopping injecting high-pressure fluid and turning off the first control valve 31 and the fourth control valve 34 to keep a stable pressure in the fracture;
then, simultaneously injecting high-pressure fluid into the second wellbore 22 of the first horizontal well and the second wellbore 23 of the second horizontal well for fracturing, and collecting fracturing data, and after the fracturing is completed, stopping injecting high-pressure fluid and turning off the second control valve 32 and the third control valve 33, so as to complete the multi-stage fracturing simulation experiment on the fan-shaped well pattern in the stratum using the synchronous fracturing mode.

The zipper fracturing mode comprises:
injecting high-pressure fluid into the first wellbore 21 of the first horizontal well for fracturing, and after the fracturing is completed, collecting fracturing data stopping injecting the high-pressure fluid, and turning off the corresponding first control valve 31;
injecting high-pressure fluid into the first wellbore 24 of the second horizontal well for fracturing, and after the fracturing is completed, collecting fracturing data, stopping injecting the high-pressure fluid, and turning off the corresponding fourth control valve 34;
injecting high-pressure fluid into the second wellbore 22 of the first horizontal well for fracturing, and after the fracturing is completed, collecting fracturing data, stopping injecting the high-pressure fluid, and turning off the corresponding second control valve 32; and
injecting high-pressure fluid into the second wellbore 23 of the second horizontal well for fracturing, and after the fracturing is completed, collecting fracturing data, stopping injecting the high-pressure fluid, and turning off the corresponding third control valve 33, so as to complete the multi-stage fracturing simulation experiment on the the fan-shaped well pattern in the stratum using the zipper fracturing mode.

In the steps of the above experimental method, only the control valves 3 described are turned on or off, and the control valves 3 not described are all considered to be in the off state.

In the experimental method for simulating fracturing of a fan-shaped well pattern according to the present disclosure, by changing the fracturing sequence of the first well bore simulator 201 and the second well bore simulator 202 in each of the well bore simulator groups 2, the synchronous fracturing mode and the zipper fracturing mode of the fan-shaped well pattern can be simulated respectively to meet different simulation requirements.

In addition, the experimental method for simulating fracturing of a fan-shaped well pattern according to the present disclosure also has the characteristics and advantages that can be achieved by the above experimental apparatus for simulating fracturing of a fan-shaped well pattern according to the present disclosure, which will not be described in detail here.

Those described above are only illustrative embodiments of the present disclosure, and are not intended to limit the scope of the present disclosure. Any equivalent change or modification made by those skilled in the art without departing from the concept and principle of the present disclosure should fall within the protection scope of the present disclosure.

What is claimed is:

1. An experimental apparatus for simulating fracturing of a fan-shaped well pattern, comprising:
a stratum simulator comprising a chamber into which materials are poured to simulate a stratum environment; and
a wellbore simulator group at least comprising a first wellbore simulator and a second wellbore simulator, wherein a first end of the first wellbore simulator and a first end of the second wellbore simulator are respectively located outside the chamber and respectively provided with a control valve, a second end of the first wellbore simulator is extended into the chamber and provided with a first perforation simulator, a second end of the second wellbore simulator is extended into the chamber and passes through the corresponding first perforation simulator, and the second end of the second wellbore simulator is provided with a second perforation simulator, so as to simulate a multi-stage fracturing scenario of the fan-shaped well pattern.

2. The experimental apparatus for simulating fracturing of a fan-shaped well pattern according to claim 1, wherein there are a plurality of the wellbore simulator groups which are independent from each other.

3. The experimental apparatus for simulating fracturing of a fan-shaped well pattern according to claim 1, wherein an included angle between the first wellbore simulator and a maximum horizontal principal stress direction and an included angle between the second wellbore simulator and the maximum horizontal principal stress direction are preset to simulate different multi-stage fracturing scenarios of the fan-shaped well pattern.

4. The experimental apparatus for simulating fracturing of a fan-shaped well pattern according to claim 1, wherein the control valve is a three-way valve, which comprises a first port, a second port and a third port, which are respectively connected to a booster pump, the wellbore simulator group and a pressure gauge.

5. The experimental apparatus for simulating fracturing of a fan-shaped well pattern according to claim 1, wherein the first perforation simulator and the second perforation simulator each is formed by two polytetrafluoroethylene sheets that are oppositely attached to each other; and
the first perforation simulator is nested at the second end of the first wellbore simulator, and the second perforation simulator is nested at the second end of the second wellbore simulator.

6. The experimental apparatus for simulating fracturing of a fan-shaped well pattern according to claim 5, wherein a direction of the first perforation simulator relative to the first wellbore simulator is adjusted, and/or a direction of the second perforation simulator relative to the second wellbore simulator is adjusted, to change an angle of a perforation formed by the two polytetrafluoroethylene sheets.

7. An experimental method for simulating fracturing of a fan-shaped well pattern, which adopts an experimental apparatus for simulating fracturing of a fan-shaped well pattern;
wherein the experimental apparatus comprises:
a stratum simulator comprising a chamber into which materials are poured to simulate a stratum environment; and
a wellbore simulator group at least comprising a first wellbore simulator and a second wellbore simulator, wherein a first end of the first wellbore simulator and a first end of the second wellbore simulator are respectively located outside the chamber and respectively provided with a control valve, a second end of the first wellbore simulator is extended into the chamber and provided with a first perforation simulator, a second end of the second wellbore simulator is extended into the chamber and passes through the corresponding first perforation simulator, and the second end of the second wellbore simulator is provided with a second perforation simulator; and
the method comprises:
step S1: applying a three-dimensional stress confining pressure to the materials poured into the stratum simulator to simulate a stratum pressure; and
step S2: sequentially injecting high-pressure fluid into the first wellbore simulator and the second wellbore simulator in the wellbore simulator group for fracturing, respectively, to simulate a multi-stage fracturing scenario of the fan-shaped well pattern in a stratum.

8. The experimental method for simulating fracturing of a fan-shaped well pattern according to claim 7, wherein in the step S1, the wellbore simulator group is connected to a booster pump and a pressure gauge through a control valve, and the simulated stratum pressure is adjusted through a cooperation of the booster pump and the wellbore simulator group, to make the simulated stratum pressure the same as an actual stratum pressure.

9. The experimental method for simulating fracturing of a fan-shaped well pattern according to claim 7, wherein in the step S2, the multi-stage fracturing scenario of the fan-shaped well pattern in the stratum is simulated using a synchronous fracturing mode, wherein the number of the wellbore simulator groups is at least two, and the synchronous fracturing mode comprises:
step S201: injecting high-pressure fluid into the first wellbore simulators in the two wellbore simulator groups for fracturing, respectively;
step S202: collecting fracturing data of the two first wellbore simulators respectively, and stopping injecting the high-pressure fluid into the two first wellbore simulators;
step S203: injecting high-pressure fluid into the second wellbore simulators of the two wellbore simulator groups for fracturing, respectively;
step S204: collecting fracturing data of the two second wellbore simulators, respectively, and stopping injecting the high-pressure fluid into the two second wellbore simulators; and
step S205: completing the multi-stage fracturing simulation experiment on the fan-shaped well pattern in the stratum using the synchronous fracturing mode.

10. The experimental method for simulating fracturing of a fan-shaped well pattern according to claim 7, wherein in the step S2, the multi-stage fracturing scenario of the fan-shaped well pattern in the stratum is simulated using a zipper fracturing mode, wherein the number of the wellbore simulator groups is at least two, and the zipper fracturing mode comprises:
step S201′: injecting high-pressure fluid into the first wellbore simulator in the first wellbore simulator group for fracturing, collecting fracturing data of the first wellbore simulator, and then stopping injecting the high-pressure fluid;
step S202′: injecting high-pressure fluid into the first wellbore simulator in the second wellbore simulator group for fracturing, collecting fracturing data of the first wellbore simulator, and then stopping injecting the high-pressure fluid;
step S203′: injecting high-pressure fluid into the second wellbore simulator in the first wellbore simulator group for fracturing, collecting fracturing data of the second wellbore simulator, and then stopping injecting the high-pressure fluid;
step S204′: injecting high-pressure fluid into the second wellbore simulator in the second wellbore simulator group for fracturing, and then stopping injecting the high-pressure fluid; and
Step S205′: completing the multi-stage fracturing simulation experiment on the fan-shaped well pattern in the stratum using the zipper fracturing mode.
